(19) United States
(12) Patent Application Publication
(10) Pub. No.: US 2010/0099080 A1
(43) Pub. Date: Apr. 22, 2010

(54) NUCLEIC ACID MEMORY DEVICE

(75) Inventors: George M. Church, Brookline, MA (US); Jay Shendure, Chagrin Falls, OH (US)

Correspondence Address: BANNER & WITCOFF, LTD., 28 STATE STREET, SUITE 1800, BOSTON, MA, 02109-1701, US

(73) Assignee: President and Fellows of Harvard College, Cambridge, MA (US)

(21) Appl. No.: 11/766,222

(22) Filed: Jun. 21, 2007

Related U.S. Application Data
(63) Continuation of application No. 10/427,745, filed on May 1, 2003, now abandoned.
(60) Provisional application No. 60/376,918, filed on May 1, 2002.

(51) Int. Cl. C12Q 1/68; C12P 19/34; C40B 50/18

(52) U.S. Cl. 435/6; 435/91.1; 506/32

(57) ABSTRACT

This invention pertains to methods of imparting information onto nucleic acid sequences. In specific embodiments, the present invention provides site-specific recombinase systems and error-prone polymerase systems to alter nucleotide sequences such as DNA and mini-genomes, as well as for the production of microarrays. The present invention also provides methods of analyzing the modified nucleotides sequences provided herein. Methods of engineering and screening for novel modified polymerases that incorporate chain terminating nucleotides and/or labeled nucleotides more efficiently than wild-type polymerases are also provided.

### RELATED APPLICATIONS

This application is a continuation of U.S. patent application Ser. No. 10/427,745, filed May 1, 2003; which claims priority from U.S. Provisional Patent Application No. 60/376,918, filed May 1, 2002; each of which is hereby incorporated by reference in its entirety for all purposes.

### STATEMENT OF GOVERNMENT INTERESTS

This invention was funded by DOE Grant No. DE-FG02-87ER60565 from the U.S. Department of Energy, and by DARPA Grant No. F3062-01-20586 from the U.S. Defense Advanced Research Projects Agency. The U.S. Government has certain rights in this invention.

### BACKGROUND OF THE INVENTION

Bio-computation is aimed at exploring and developing computational methods and models at the bio-molecular and cellular levels. However, this field has focused on computational algorithms rather than Input/Output (I/O) and memory. For example, present DNA-enzyme clock rates are typically six logs slower that the GHz expected of electronic-optical (EO) computing. Accordingly, input, output and memory options are needed.

### SUMMARY OF THE INVENTION

The present invention is directed to the use of nucleic acid polymers, whether fully- or partially single-stranded, double-stranded, or multi-stranded, as storage media for memory. Information can be recorded onto the nucleic acid polymers in vivo or in vitro. The nucleic acid polymers include single bit or multiple bit information depending on their design. According to one embodiment of the invention, the nucleic acid polymers are included on a support substrate whether in an ordered or random manner.

The nucleic acid polymers can be altered, i.e. information written onto the nucleic acid polymers, using, for example, site-specific recombinases to alter the position of a nucleic acid segment or otherwise eliminate a nucleic acid segment. Site-specific recombinases can also be used to synthesize arrays of nucleic acids on substrates. In addition, polymerases, including without limitation error-prone template-dependent polymerases, modified or otherwise, can be used to create a nucleic acid polymer having the desired information thereon. Template-independent polymerases, whether modified or otherwise, can be used to create a nucleic acid polymer de novo, which polymer carries stored information. Such polymerases can be used to incorporate reversible chain terminating nucleotides and/or labeled nucleotides into a nucleic acid sequence at an increased efficiency over wild-type polymerases and also to synthesize arrays of nucleic acids on substrates. Sensors, such as light activated sensors, metabolic products or chemicals, that are activated by ligands can be used with such polymerases.

Information can be read from the nucleic acid polymers by interrogating the nucleic acid polymers using, for example, detectable tags or hybridization or amplification methods including fluorescent in situ sequencing, rolling circle amplification and others. Additional methods of interrogating nucleic acid arrays are described in U.S. Pat. No. 6,326,489 incorporated herein by reference.

Combinations of features and methods described herein are also provided.

### BRIEF DESCRIPTION OF THE DRAWINGS

FIG. 1 depicts a schematic representation of a construct encoding four central portions, wherein each central portion is located between two site-specific recombinase (SSR) binding domains (set forth as arrows). The SSR binding domains are oriented in anti-parallel.

FIG. 2 depicts a schematic representation of DNA memory using DNA polymerases. This figure illustrates that an etaDNAPol/RNAPo1 fusion polymerase can only advance effectively if the next dNTP and rNTP in line are both present.

FIG. 3 depicts a schematic representation of Rolling Circle Amplification (RCA).

FIG. 4 depicts a schematic representation of a three dimensional array.

### DETAILED DESCRIPTION OF CERTAIN PREFERRED EMBODIMENTS

Embodiments of the present invention are based on the discovery of novel methods by which information can be imparted into nucleic acid sequences such as DNA molecules. The nucleic acid sequences can be collected or are arranged onto a substrate in an ordered or random manner. The information may be recorded in a nucleic acid sequence in vivo, e.g., in or on the cell surfaces of bacteria, viruses, tissue culture cells, multicellular organisms and the like, or in vitro, e.g., on microarrays, beads, slides, test tubes and the like.

According to one aspect of the present invention, site-specific recombinases are used to alter a nucleic acid sequence in a manner to impart information to the nucleic acid sequence. This process is analogous to writing information onto storage or memory media. Site-specific recombinases (SSRs) are a family of enzymes that catalyze specific rearrangements of DNA (Nash (1996) “Site-specific recombination: integration, excision, resolution, and inversion of defined DNA segments,” pp. 2363-2367 In Escherichia coli and Salmonella typhimurium, vol. 1. ASM, Washington, D.C.). Broadly speaking, SSRs are capable of catalyzing three such reactions: integration, excision, and inversion. The relative orientation and location of the SSR binding sites specifies the particular rearrangement that the enzyme catalyzes. In addition, homologous recombination in general using for example lambda red, recA and other homologous recombination enzymes are useful in the present invention as they mediate homologous recombination but may not recognize a specific site.

SSRs are commonly employed in experiments aimed at cell lineage analysis and/or the study of tissue-specific gene function in multi-cellular organisms. Generally, specific expression of an SSR (generally Flp or Cre, two particularly well-characterized SSRs) catalyzes an excision event of the DNA intervening (referred to herein as the “central portion”) two SSR binding sites (frt or loxP, respectively) that are oriented in parallel. Enzymes such as AID and CSR are also known to catalyze excision events (Yatabe et al. (2001) Proc. Natl. Acad. Sci. USA 2001 98(19):10839-44; Okazaki et al. (2002) Nature 416:340-345; Santoro et al. (2002) Proc. Natl. Acad. Sci. USA 99: 4185-4190) The central portion of an SSR binding site, its “spacer,” is thought to be generally unimportant for SSR activity, but critical for the matching of any two sites between which the SSR will catalyze a recombination event. The enzymatic mechanism of SSR action depends on homology pairing of the spacers of two SSR binding sites. Small differences between the spacer sequences of a given pair of SSR binding sites can drastically reduce the frequency of recombination events between those sites. Thus, directed mutations can result in an SSR binding site that undergoes reactions with the wild-type binding site at a drastically reduced frequency, but the site is still functional, in that it can react efficiently with a binding site that contains an identical set of mutations. Directed changes in the SSR binding sites, such as those disclosed in Sauer (1996) Nucleic Acids Res. 24(23):4608-4613; Schlake (1994) Biochemistry 33(43):12746-12751 can be used to “insulate” sites from one another, permitting multiple non-interacting genomic manipulations with a single SSR system.

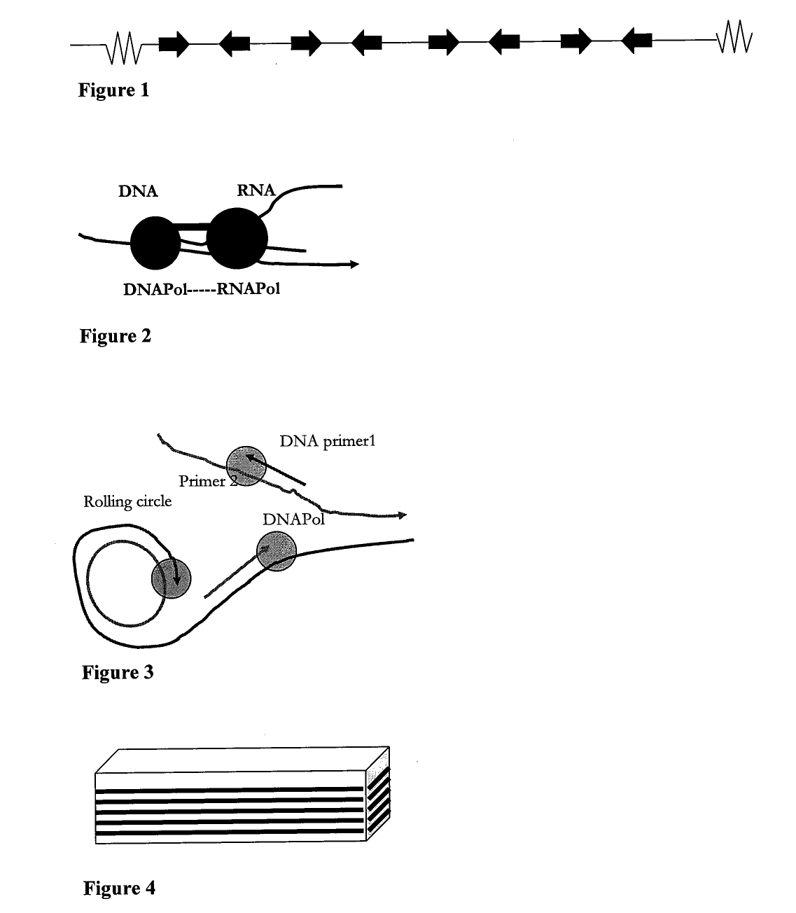

The two possible catalytic activities of an SSR on two binding sites located on a single strand of DNA (excision or inversion) can be abstracted as discreet transitions between two potential states of a single bit or unit of information equal to one binary decision. A pair of sites oriented in parallel can only experience an irreversible transition event (0->1), namely, excision. Sites oriented in anti-parallel, on the other hand, can experience reversible transition events (0<->1), as the intervening DNA flips from one orientation to the other in the presence of an SSR. If more than one of such bits (each consisting of a pair of sites), all on the same DNA construct, can be efficiently insulated from reacting with one another (as discussed above), the number of potential states of such a construct would rise exponentially with the number of two-state bits available. A byte, for example, consisting of 8 bits, is capable of 256 states (2̂8). A DNA construct of 30 insulated bits, each consisting of a pair of interacting SSR binding sites, would theoretically be capable of existing in 2̂30 (˜1 billion) different states. Linear increases in the size of the DNA construct would result in exponential gains in the number of potential states. Read-out of the bit states can be accomplished via direct sequencing, directional PCR, or probe hybridization. A schematic of a multi-state construct with reversible bits (SSR binding sites oriented in anti-parallel) is set forth in FIG. 1. In one embodiment, in vivo recombination can be linked to known sensor pathways. Sensor pathways are reviewed in Dymecki ((1996) Gene 171 (2) : 197-201), Sabath et al. ((2000) Biotechniques 28(5):966-72, 974), and Srinivas et al. ((2001) BMC Dev. Biol. 1(1):4), incorporated herein by reference in their entirety.

In a preferred embodiment, a plasmid-based two-bit construct using two pairs of Flp binding sites oriented in anti-parallel is provided. This construct is consequently capable of four states (2̂2). State-switching is reversible and essentially random in the presence of the Flp recombinase. Differences in the switching rates of the two bits differ from one another. The bits are relatively insulated from one another, as restriction enzyme analysis of plasmids exposed to Flp does not reveal a significant quantity of excision events between elements of the different bits. In another embodiment, the Flp enzyme is placed under the control of more tightly regulatable promoters generating two constructs, each with a greater number of bits (one with reversible bits and the other with irreversible bits), cloning the multi-bit construct into a BAC (bacterial-artificial-chromosome) in a BAC-compatible strain, where the construct is to be present in low copy number (one or two copies) and wild-type recombination systems are knocked out.

According to one embodiment, a relatively low number of bits (e.g., 50) can encode a very high number of states (10̂15). This aspect of the invention coupled with a mechanism of stochastic flipping at a low rate provides a useful method for high-throughput multiplexed cell lineage analysis of multicellular organisms (or monitoring world-wide transport of tagged biomaterials in general). The system provides, essentially, a unique tracking code that evolves over cell generations or over time. In one embodiment 50, 45, 40, 35, 30, 25, 20, 15, 10, 5 or less bits are provided. In another embodiment 50, 55, 60, 65, 70, 75, 80, 85, 90, 95, 100, 125, 150, 175, 200, 225, 250, 275, 300, 325, 350, 375, 400, 425, 450, 475, 500, 600, 700, 800, 900, 1000 or more bits are provided. As a transgenic organism (carrying the multi-bit construct and expressing the Flp enzyme) develops, the bit-state of any given cell lineage would change. As cell lineages diverge from one another, so would the bit-states. The bit-state profile of any given cell is informative of the degree to which the SSR was expressed in its ancestors, as well as of its phylogenetic relationship to other cells with similar profiles. In a preferred embodiment, the same sorts of mathematical methods developed for the determination of evolutionary phylogeny on the basis of sequence data is used to analyze these data. In another preferred embodiment, the Yatabe methylation lineage concept is adapted for in situ sequencing.

In one embodiment, the Flp enzyme is placed under the control of any promoter, and the activation of specific transcriptional programs is “recorded” in investigations where direct observation of a reporter gene is not a tractable option. For example, transcriptional programs involving anaerobic growth activated during the in vivo infection by Pseudomonas aeruginosa may be monitored. The use of a multi-state construct using irreversible bits that undergo transitions with varying efficiencies which depend on the cellular concentration of the FLP enzyme provide a quantitative record of the extent of activation achieved under a given transcriptional program and can be recorded.

According to an additional aspect of the present invention, DNA is used to perform computations or store information which would benefit from a read-out method. Information is encoded in the smallest, most accurately replicated bits in nature, the base pairs themselves. For example, specific changes in DNA are replicated and can be assayed at any subsequent time point. In one aspect, the use of SSR binding site-based bits, using natural or engineered SSRs, serve as a recording method for DNA and/or cellular computing technologies. In another aspect, multiple independent sensors are run simultaneously by harvesting the hundreds of different recombinase site-specificities in various microbial species to make combinations (Shaikh (2000) J. Mol. Biol. 302(1):27-48) or select for variants (Bulyk (2001) Proc. Nat. Acad. Sci. USA 98: 7158-7163).

According to an alternate embodiment of the present invention, polymerases are used to build nucleic acid molecules representing recorded information. Polymerases are enzymes that produce a nucleic acid sequence, for example, using DNA or RNA as a template. Polymerases that produce RNA polymers are known as RNA polymerases, while polymerases that produce DNA polymers are known as DNA polymerases. Polymerases that incorporate errors are known in the art and are referred to herein as an “error-prone polymerases” or “template-independent polymerases”. Error-prone polymerases will either accept a non-standard base, such as a reversible chain terminating base, or will incorporate a different nucleotide that is selectively given to it as it tries to copy a template. Template-independent polymerases such as TdT create nucleic acid strands without a template. By limiting nucleotides available to the polymerase, the incorporation of specific nucleic acids into the polymer can be regulated. Thus, these polymerases are capable of incorporating nucleotides independent of the template sequence and are therefore beneficial for creating nucleic acid sequences de novo. The combination of an error-prone polymerase and a primer sequence serves as a writing mechanism for imparting information into a nucleic acid sequence. Methods of limiting nucleic acid availability are discussed further below.

The eta-polymerase (Matsuda et al. (2000) Nature 404(6781):1011-1013) is an example of a polymerase having a high mutation rate (˜10%) and high tolerance for 3′ mismatch in the presence of all 4 dNTPs and probably even higher if limited to one or two dNTPs. Hence, the eta-polymerase is a de novo recorder of nucleic acid information similar to terminal deoxynucleotidyl transferase (TdT) but with the advantage that the product produced by this polymerase is continuously double-stranded. Double stranded DNA has less sticky secondary structure and has a more predictable secondary structure than single stranded DNA. Furthermore, double stranded DNA serves as a good support for polymerases and/or DNA-binding-protein tethers.

In a preferred embodiment, the present invention provides novel mutated or engineered polymerases that incorporate chain terminating nucleotides and/or labeled nucleotides. Mutated polymerases may be those that occur in nature or otherwise spontaneously. Alternatively, mutations may be intentionally induced, such as by subjecting an organism, cell or cell-free system to mutagenic conditions (including, in non-limiting fashion, radiation) or compositions. Mutagenic compositions, which include without limitation EMS and MMS, are well known in the art. Preferably, the chain terminating nucleic acids are reversible terminators. Reversible terminators are discussed further below. In a preferred embodiment, novel polymerases are identified or engineered and polymerases are selected based on their increased ability to incorporate reversibly 3′ blocked dNTPs (e.g., nitrobenzyl or NPPOC), and/or labeled nucleotides. In a preferred embodiment, photocleavable blocking groups are used in association with microarray mirrors such as those available from Nimblegen to allow rapid and easily patterned array manufacture. It has been shown that substitution of specific amino acid residues, such as the polar uncharged residues cysteine, serine and asparagines at residue 562 of T7 DNA polymerase can reduce its activity by 10- to 50-fold to incorporate dideoxynucleotides into the oligonucleotide chain (Tabor and Richardson (1995) Proc. Natl. Acad. Sci. USA 92:6339). These substitutions can also be made with the 3′ blocked dNTPs in contrast to currently available DNA polymerases. Further Tet/z polymerase can be used to incorporate 3′ alkyl ethers. J. Chem. Soc. Perkin Trans 1994. In addition, the homologous amino acid positions in other polymerases (e.g., a terminal transferase enzyme) are suitable for light-directed stepwise DNA synthesis and for mutating in order to engineer polymerases having an increased ability to incorporate reversible terminators and/or labeled nucleotides. The mutant polymerases are also useful as a starting point for screening large libraries of in-vitro-protein-synthesized polymerases for fluorescent dNTP incorporation.

A particularly preferred polymerase for screening is the human immunodeficiency virus (HIV) polymerase reverse transcriptase (RT). Mutant polymerases are selected using screening assays provided herein. In a further embodiment, polymerase mutants are one starting point for screening large libraries of polymerases synthesized by in vitro translation for their ability to incorporate fluorescent dNTPs into a polynucleotide sequence. Using this method, one of skill in the art could identify mutants that are toxic or unstable in vivo.

Mutant polymerases of the invention preferably lack 3′-5′ exonuclease activity and other undesired chemical and enzymatic releasing activities that would release 3′ blocking groups, as normally stable protecting groups (such as thioureas and acetals) can be rapidly removed by polymerases. The incorporation of 3′ alkyl ethers with the Tet/z polymerase has also been observed (Lonberg (1994) J. Chem. Soc. Perkin Trans.).

In another embodiment, the present invention provides an error-prone DNA polymerase attached to an RNA polymerase. The fused polymerase which may operate with or without a template is useful for nano-positioning devices in general, analogous to piezo-positioning in scanning tunnel microscopy but more highly parallel, compact and bio-compatible. A fused DNA-RNA polymerase is discussed further below.

Particular embodiments of the present invention utilize reversible chain-terminating nucleotides. As used herein, the term “reversible” refers to a blocking group that, when present on an NTP, prevents further 3′ nucleic acid polymer synthesis. An example of reversible chain terminating nucleotides are reversibly 3′ blocked dNTPs. Upon removal of the blocking group, additional nucleotides can be attached to the nucleotide at the 3′ end of the polymer using, for example, the polymerases described herein. Preferably, the reversible chain terminating nucleotides of the invention are nucleotides having photo-cleavable blocking groups (e.g., nitrobenzyl or NPPOC). Chemically-cleavable blocking groups are also embraced by the present invention. Reversible terminators are known in the art and are disclosed in U.S. Pat. Nos. 6,087,095, 6,255,475, 6,309,836, incorporated herein by reference in their entirety.

Nucleotides and/or primers of the invention may be labeled in a variety of ways, including the direct or indirect attachment of radioactive moieties, fluorescent moieties, colorimetric moieties, and the like. Examples of useful labels include, but are not limited to fluorescein, luciferase, Texas red, yellow fluorescent protein (YFP), green fluorescent protein (GFP), cyan fluorescence protein (CFP), BODIPY, dansyl, rhodamine, Cy 2, Cy 4, Cy 6, and the like.

Many comprehensive reviews of methodologies for labeling DNA are available (see Matthews et al., 1988, Anal. Biochem., 169: 1-25; Haugland, 1992, Handbook of Fluorescent Probes and Research Chemicals, Molecular Probes, Inc., Eugene, Oreg.; Keller and Manak, 1993, DNA Probes, 2nd Ed., Stockton Press, New York; Eckstein, ed., 1991, Oligonucleotides and Analogues: A Practical Approach, ML Press, Oxford, 1991); Wetmur, 1991, Critical Reviews in Biochemistry and Molecular Biology, 26: 227-259). Many more particular labeling methodologies are known in the art (see Connolly, 1987, Nucleic Acids Res., 15: 3131-3139; Gibson et al. 1987, Nucleic Acids Res., 15: 5455-6467; Spoat et al., 1987, Nucleic Acids Res., 15: 4837-4848; Fung et al., U.S. Pat. No. 4,757,141; Hobbs, et al., U.S. Pat. No. 5,151,507; Cruickshank, U.S. Pat. No. 5,091,519; (synthesis of functionalized oligonucleotides for attachment of reporter groups); Jablonski et al., 1986, Nucleic Acids Res., 14: 6115-6128 (enzyme/oligonucleotide conjugates); and Urdea et al., U.S. Pat. No. 5,124,246 (branched DNA)). Each of these references is incorporated herein in its entirety for any and all purposes.

To accomplish the recording of the levels of one or more of the dNTPs, they must be regulated directly or indirectly by the environmental components to be recorded. Various degradative, biosynthetic, or salvage nucleotide pathways can be controlled in vitro via a subset of sensors. Examples for each dNTP degradation have been characterized—dCTPases (e.g., phage T4), dATPases (e.g., helicases), dUTPases (e.g., Dut), dGTPases (e.g., E. coli MutT), and TTPases (Shechter (2000) J. Biol. Chem. 275(20):15049-15059; Frick, et al. (1994) J. Biol. Chem. 269(3):1794-1803; Baldo (1999) J. Virol. 73(9):7710-7721; Gary et al. (1998) Genetics 148(4):1461-1473; Schultes et al. (1992) Biol. Chem. Hoppe Seyler 373(5):237-247). In an alternative embodiment, photoactivated dNTPs and/or chemically-activated primers are used separately, or combined with any of the pathways set forth above. Preferred recordings include but are not limited to light, stress, toxins, and/or glucose monitoring. A preferred standard (initial) readout is conventional electrophoretic sequencing or “in situ” DNA sequencing. In one aspect, the initial applications use variable length (unclocked) nucleotide runs. In another aspect, increasingly accurately phased syntheses are performed using appropriate combinations of polymerases and dNTP concentrations. For example, an RNA polymerase fused to the eta polymerase could act as a stepper-positioner moving a precise number of by based on a sequence of rNTPs. In yet another aspect, partial overwriting of previous messages is used. FIG. 2 illustrates that the etaDNAPol and RNAPo1 can only advance (effectively) if the next dNTP and rNTP in line are both present.

In another preferred embodiment, the DNA synthesis (recording) alternates from mismatch to perfect match so that polymerases that only allow one mismatch a time are employed. In another embodiment the DNA synthesis is done by multiple passes, i.e., first strand then second then optionally first strand again, etc. to allow high fidelity synthesis.

In another embodiment, the polymerase-memory is used to record images or chemical time-courses in very compact form (1 nm³ per bit) analogous to flight-recorders and other devices where much more information is recorded than is played back. In another embodiment, the polymerase-memory is used for synthesis of long-DNA molecules.

According to an alternate embodiment of the invention, small ligands are used to input data into engineered cellular and in vitro systems. Currently, there are 58 DNA-binding proteins adapted to E. coli with known ligand/inducers and generally non-cross-reacting DNA-specificity (Robison (1998) J. Mol. Biol. 284:241-254). Another set of sensors depends on termination control (His, Trp, Ile, Val, Thr, Phe, etc.). In a preferred embodiment, these small ligands, which allow programming from the outside of the cellular systems, are provided. In another embodiment, multiple regulatory molecules in vivo are provided. For the in vitro system, a complete empirical code for Zn-fingers and DNA-binding domains in general using phage display and double stranded DNA (ds-DNA) microarrays is provided (Bulyk (2001)). This provides a general method for long-term fine-tuning of individual promoters by creating concentration gradients of the appropriate DNA binding proteins. This is considerably more cost-effective than synthesis of a series of DNA constructs and allows determination of additional network parameters usable in modeling. Allosteric repressors have been used to regulate T7 RNA polymerase (Dubendorff (1991) J. Mol. Biol. 219(1):45-59). In one aspect of the invention, small molecules are used to trigger allosteric ribozyme switches (Seetharaman (2001) Natl. Biotechnol. 19(4):336-341). In one embodiment the cAMP-triggered ribozyme is inserted in frame at the 5′ end of GFP such that upon cAMP-induced cleavage the GFP mRNA becomes more (or less) translatable.

According to one aspect of the present invention, light is used to record complicated patterns in long DNA molecules. Currently, photocleavable phosphoramidites are spatially patterned by light projected from megapixel micro-mirror arrays (available from Texas Instruments) (Singh-Gasson (1999) Natl. Biotechnol. 17(10):974-978) for organic synthesis. Photocleavable nucleotide triphosphates (nitro-benzyl “caged” NTPs) have been used in cell physiology (McCray (1980) Proc. Natl. Acad. Sci. USA 77(12):7237-7241; Densham, EP 1165786, AU 4382801, AU 735898, AU 7546700, WO 0125480). In a preferred embodiment these two approaches are combined with the polymerase-recording concept set forth above. In one aspect, a fluorescent nucleotide triphosphate is incorporated by T7 RNA polymerase under control of a caged ATP or GTP. In another aspect these two approaches are used to regulate the incorporation of dNTPs in the format of an RNAPo1-etaDNAPol fusion.

In a particularly preferred embodiment the cellular system processes its small ligand inputs, and subsequently records its computations on one or more DNA molecules. Because this system is capable of generating large amounts of data (billions of bits), high throughput methods of sequencing these DNA molecules, such as that disclosed in Mitra (1999) Nucleic Acids Res. 27(24):e34; pp. 1-6, are useful. In preferred embodiments, high throughput methods are used with PCR amplicons or other nucleic acid molecules having lengths of less than 100 bp. In other preferred embodiments, PCR amplicons of 100 bp, 110 bp, 120 bp, 130 bp, 140 bp, 150 bp, 160 bp, 170 bp, 180 bp, 190 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 by or more may be used. In one aspect, this method is used to assess the patterns produced for spatial and nucleotide fidelity. Using an error-prone polymerase allows the incorporation of specific bases at precise locations of the DNA molecule. Thus, eta polymerase with 10% error in the presence of all 4 dNTPs could be close to 99% “error” if given only one non-template-matching dNTP. This would effectively mean 1% error in the specified goal. The fidelity of incorporation for a variety of high-fidelity polymerases when presented with only one dNTP has been measured. The present invention provides for repeating such quantitations using the eta polymerase or other error-prone polymerase. The present invention further provides for testing chemosensor recording “black-boxes” in which arrays of allosteric self-cleavage induced by specific small ligands, e.g., cAMP (Seetharaman (2001)), will produce active RNA primers for initiating DNA synthesis.

Rolling Circle Amplification (RCA) (Zhong (2001) Proc. Natl. Acad. Sci. USA 98(7):3940-3945) represents an alternative to polony amplification since it is continuous replication and does not require thermal cycling. With only one primer (or nick), it grows one long tail (set forth in FIG. 3) from the original circle at a rate linear with time. Isothermal amplification of a circular or linear nucleic acid template also can be performed according to Tabor and Richardson (WO 00/41524) using methods in which enzymatic synthesis of nucleic acid molecules occurs in the absence of oligonucleotide primers.

When a second primer from the opposite strand is also included, —highly branched structures are produced, —with mass growing initially exponentially with respect to time (m=k*exp(t), or at least m=kt²).

Modeling of the RCA process described herein indicates a novel way to build up layers in a 3D array as a function of time, chemicals and optical patterns (set forth in FIG. 4). If replication begins in a uniform layer on the flat surface of a glass slide (or other surface), then the polymerization reaction can only occur in the next nm thick layer up. The strand-displacing activities of polymerases (such as etaPol or an eta-like BstPol) in RCA requires either nicks or primers to initiate strand-displacing DNA synthesis (see FIG. 3). If some of the RCA primers are immobilized then the hyperbranched-DNA products will be quite stable in space and time. A coarse (micron-scale, 5 Hz) pattern can be set by the megapixel micro-mirror optics, while finer detail (m, 250 Hz) is provided by either a free running or RNAPol-etaDNAPol-fusion stepper. Nano-scale recording is not necessarily “redundant” nor limited by the micron scale light patterns, since it contains time components. The thickness, and therefore the recording capacity, would be effected by the spatiotemporal precision of specific NTP and/or dNTP pulses used for positioning and recording respectively.

In a preferred embodiment, the layers deposited can include a variety of chemistries attached to (or placed by) the nucleic acids. In one aspect, redox-sensitive fluorophore “side-chains” have been developed for each of the four dNTPs. In another aspect, photosensitive versions of each of the four dNTPs can be developed using methods known to those of skill in the art (Rob Mitra, unpublished data (2000)). In yet another aspect, metal binding groups and wires (Braun (1998) Nature 391(6669):775-778), quantum dots (Michler (2000) Nature 406(6799):968-70), quantum-wires (Emiliani (2001) J. Microsc. 202(Pt 1):229-240), magnetic dots (Cowburn (2000) Science 287(5457):1466-1468), or refractive dots (Yguerabide (1998) Anal. Biochem. 262(2):157-76), can be assembled by this method. The 3D arrays of the present invention provide fast electronic-optical pathways based on signal coincidences and/or traffic levels (analogous to learning and computing in neural circuits). The naturally hyperbranched structures found in RCA are an elegant first step in this direction.

Nucleic acid arrays can be manufactured by a variety of methods including those disclosed in U.S. Pat. No. 6,326,489 hereby incorporated by reference in its entirety. According to one method, nucleic acid arrays can be synthesized by first immobilizing single-stranded DNA molecules to the surface of a solid support followed by priming and enzymatic synthesis of a second nucleic acid strand, either RNA or DNA. A highly preferred method of carrying out synthesis of the immobilized single-stranded array is presented in U.S. Pat. No. 5,556,752.

In addition, light-directed methods can also be used to make nucleic acid arrays. Such light-directed array making methods are described in U.S. Pat. No. 5,143,854, U.S. Pat. No. 5,510,270 and U.S. Pat. No. 5,527,681 hereby incorporated by reference in their entireties. These methods involve activating predefined regions of a substrate or solid support and then contacting the substrate with a preselected monomer solution. these regions can be activated with a light source, typically shown through a mask (much in the manner of photolithography techniques used in integrated circuit fabrication. Other regions of the substrate remain inactive because illumination is blocked by the mask and they remain chemically protected. Thus, a light pattern defines which regions of the substrate react with a given monomer. By repeatedly activating different sets of predefined regions and contacting different monomer solutions with the substrate, a diverse array of polymers is produced on the substrate. Other applicable methods include mechanical techniques such as those described in U.S. Pat. No. 5,384,261 hereby incorporated by reference. Still further techniques include bead based techniques such as those described in PCTUS/93/04145 incorporated by reference and pin based methods such as those described in U.S. Pat. No. 5,288,514 also incorporated herein by reference. Still further techniques include spotting or flow channel techniques described in U.S. Pat. No. 5,384,261 hereby incorporated by reference.

Aspects of the invention are further provided where modified molecules are synthesized or arranged on an array. In one embodiment, the methods of the present invention utilize a maskless array technology whereby nucleotide sequences or single nucleotides having photocleavable groups as described above are activated by exposure to light. A preferred maskless array is a micromirror array (such as the arrays used by NimbleGen™) which employs a solid-state array of miniature aluminum mirrors to pattern up to 786,000 individual pixels of light to create a “virtual mask.” The virtual mask thus replaces a physical masks used in traditional arrays. These virtual masks reflect the desired pattern of UV light with individually addressable aluminum mirrors controlled by a computer.

Traditional methods of physically masking arrays that are known in the art are also useful for methods of the present invention (such as masking protocols used by Affymetrix™). Methods that could be used to spatially partition template molecules on a substrate which would be useful in the present invention further include those of Chetverin et al., Kawashima et al. (WO 98/44151, filed Apr. 1, 1998, published Oct. 8, 1998, incorporated herein in its entirety by reference), Adams & Kron (U.S. Pat. No. 5,641,658, issued Jun. 24, 1997, incorporated herein in its entirety by reference), two-phase (i.e., oil-in-water or water-in-oil) emulsion methods are known which permit the trapping of one phase (and any molecules dissolved or particles suspended therein) as microencapsulated droplets. These constructs may be composed of a hardened impermeable outer shell with a liquid interior (Tate, U.S. Pat. No. 4,211,668, Jul. 8, 1980, and Vassiliades U.S. Pat. No. 4,273,672, Jun. 16, 1981, incorporated herein in their entirety by reference) or may be composed of a permeable gel matrix such as agarose (Weaver et al. (1991) Bio/Technology 9: 873-876, incorporated herein in its entirety by reference). The latter has been used to isolate individual metaphase chromosomes in 20-90 micron diameter microdroplets for in situ hybridization and FACS analysis (Nguyen (1995) Cytometry 21:111-119, incorporated herein in its entirety by reference). This method is of use according to the present invention. Since unattached beads do not have the benefit of a fixed Cartesian address, they are not easily re-probed in a serial manner, unless a unique identifier, a “barcode,” can be attached or incorporated within. Combinatorially encoded mixtures of dyes such as “quantum dots” are known which could be employed to give beads a unique “real estate address” (Weiss et al., U.S. Pat. No. 5,590,479, issued Nov. 23, 1999; Chandler and Villacorta, WO 01/13119 A1, filed Aug. 17, 2000, published Feb. 22, 2001; and Han et al. (2001) Nat. Biotech. 19:631-5, incorporated herein in their entirety by reference). Alternatively, once formed, the beads could be deposited and fixed on a surface or immobilized in a flow cell using methods such as those taught by Lynx Therapeutics, where the beads can be serially re-probed with no loss of spatial address (Brenner (2000) Nature Biotechnology 18: 630-634, incorporated herein in its entirety by reference).

The method of haplotyping by Single Molecule Dilution in conventional microtiter plates (Stephens et al. (1990) Am. J. Hum. Genet. 46:1149-1155; Ruano et al. (1990) Proc. Natl. Acad. Sci. U.S.A. 87: 6296-300); and Vogelstein and Kinzler (1999) Proc. Natl. Acad. Sci. U.S.A. 96:9236-41, “digital PCR,” incorporated herein in their entirety by reference) can be greatly miniaturized and thereby economized with fabricated devices that permit multiplex handling of many small pools of liquids in volumes less than 100 nanoliters. Systems for analyzing a plurality of liquid samples consisting of a platen with two parallel planar surfaces and through-holes dimensioned to maintain a liquid sample in each through-hole by surface tension are known in the art (EP 1051259A1, Nov. 15, 2000, incorporated herein in its entirety by reference). Samples can be drawn from a planar surface using capillary action and can be diluted and mixed. Each through-hole can be queried by optical radiation. This device, as well as ones like it such as the Flow-Thru Chip™ of Gene Logic (Torres et al., WO 01/45843 A2, Jun. 28, 2001, incorporated herein in its entirety by reference), is of use according to the current invention. The inner walls of each chamber can be functionalized with 5′-attached template nucleic acid sequences and all the other necessary reagents (such as site-specific recombinases or error-prone polymerases and nucleotides) are delivered in liquid phase to each discrete chamber (or “honeycomb” cell).

In certain embodiments, substrates such as microscope slides can be separated 1) by a wettable surface boundary area if the same pool of analyte nucleic acid molecules is intended to be evenly spread across all features on a slide or 2) by a non-wettable surface boundary area if each feature is to be spotted with a different pool of analyte nucleic acid molecules and/or primers. Combinations of the above are also possible, such as slides subdivided into larger groups of continuously wettable areas, each bounded by a non-wettable boundary, where each wettable area is further divided into smaller features each bearing different spotted primers.

According to another embodiment, it is also possible to compartmentalize single DNA molecules by dipping a slide possessing small discontinuous hydrophilic features separated by a continuous hydrophobic boundary into an aqueous solution of dilute DNA template molecules. As the slide is removed and gently blotted on its side, small beads of liquid will form over the hydrophilic features, thereby creating small discontinuous pools of liquid bearing 0, 1 or >=2 DNA template(s) (See Brennan, U.S. Pat. No. 6,210,894 B1, Apr. 3, 2001, incorporated herein in its entirety by reference, for a description of related art).

According to certain embodiments, a substrate is provided which supports the synthesis of the nucleic acid segments and/or supports the attachment of nucleic acid templates. Supports according to the present invention include solid supports such as those made from glass or other materials known to those skilled in the art. Examples include simple glass slides or beads. Solid supports can also include the surfaces of cells such as viruses, bacteria, or tissue culture cells. The solid supports can also include a semi-solid support such as a compressible matrix with both a solid and a liquid component, wherein the liquid occupies pores, spaces or other interstices between the solid matrix elements. Preferably, the semi-solid support materials include polyacrylamide, cellulose, poly dimethyl siloxane, polyamide (nylon) and cross-linked agarose, -dextran and -polyethylene glycol. Solid supports and semi-solid supports can be used together or independent of each other.

Supports of the present invention can be any shape, size, or geometry as desired. For example, the support may be square, rectangular, round, flat, planar, circular, tubular, spherical, and the like.

Supports can also include immobilizing media. Such immobilizing media that are of use according to the invention are physically stable and chemically inert under the conditions required for nucleic acid molecule deposition and amplification. A useful support matrix withstands the rapid changes in, and extremes of, temperature required for PCR. The support material permits enzymatic nucleic acid synthesis. If it is unknown whether a given substance will do so, it is tested empirically prior to any attempt at production of a set of arrays according to the invention. According to one embodiment of the present invention, the support structure comprises a semi-solid (i.e., gelatinous) lattice or matrix, wherein the interstices or pores between lattice or matrix elements are filled with an aqueous or other liquid medium; typical pore (or ‘sieve’) sizes are in the range of 100 μm to 5 nm. Larger spaces between matrix elements are within tolerance limits, but the potential for diffusion of amplified products prior to their immobilization is increased. The semi-solid support is compressible. The support is prepared such that it is planar, or effectively so, for the purposes of printing. For example, an effectively planar support might be cylindrical, such that the nucleic acids of the array are distributed over its outer surface in order to contact other supports, which are either planar or cylindrical, by rolling one over the other. Lastly, a support material of use according to the invention permits immobilizing (covalent linking) of nucleic acid features of an array to it by means known to those skilled in the art. Materials that satisfy these requirements comprise both organic and inorganic substances, and include, but are not limited to, polyacrylamide, cellulose and polyamide (nylon), as well as cross-linked agarose, dextran or polyethylene glycol.

One embodiment is directed to a thin polyacrylamide gel on a glass support, such as a plate, slide or chip. A polyacrylamide sheet of this type is synthesized as follows. Acrylamide and bis-acrylamide are mixed in a ratio that is designed to yield the degree of crosslinking between individual polymer strands (for example, a ratio of 38:2 is typical of sequencing gels) that results in the desired pore size when the overall percentage of the mixture used in the gel is adjusted to give the polyacrylamide sheet its required tensile properties. Polyacrylamide gel casting methods are well known in the art (see Sambrook et al., 1989, Molecular Cloning. A Laboratory Manual, 2nd Edition, Cold Spring Harbor Laboratory Press, Cold Spring Harbor, N.Y., incorporated herein in its entirety by reference), and one of skill has no difficulty in making such adjustments.

The gel sheet is cast between two rigid surfaces, at least one of which is the glass to which it will remain attached after removal of the other. The casting surface that is to be removed after polymerization is complete is coated with a lubricant that will not inhibit gel polymerization; for this purpose, silane is commonly employed. A layer of silane is spread upon the surface under a fume hood and allowed to stand until nearly dry. Excess silane is then removed (wiped or, in the case of small objects, rinsed extensively) with ethanol. The glass surface which will remain in association with the gel sheet is treated with y-methacryloxypropyltrimethoxysilane (Cat. No. M6514, Sigma; St. Louis, Mo.), often referred to as ‘crosslink silane’, prior to casting. The glass surface that will contact the gel is triply-coated with this agent. Each treatment of an area equal to 1200 cm² requires 125 μl of crosslink silane in 25 ml of ethanol. Immediately before this solution is spread over the glass surface, it is combined with a mixture of 750 μl water and 75 μl glacial acetic acid and shaken vigorously. The ethanol solvent is allowed to evaporate between coatings (about 5 minutes under a fume hood) and, after the last coat has dried, excess crosslink silane is removed as completely as possible via extensive ethanol washes in order to prevent ‘sandwiching’ of the other support plate onto the gel. The plates are then assembled and the gel cast as desired.

The only operative constraint that determines the size of a gel that is of use according to the invention is the physical ability of one of skill in the art to cast such a gel. The casting of gels of up to one meter in length is, while cumbersome, a procedure well known to workers skilled in nucleic acid sequencing technology. A larger gel, if produced, is also of use according to the invention. An extremely small gel is cut from a larger whole after polymerization is complete.

Note that at least one procedure for casting a polyacrylamide gel with bioactive substances, such as enzymes, entrapped within its matrix is known in the art (O′Driscoll, 1976, Methods Enzymol., 44: 169-183, incorporated herein in its entirety by reference). A similar protocol, using photo-crosslinkable polyethylene glycol resins, that permit entrapment of living cells in a gel matrix has also been documented (Nojima and Yamada, 1987, Methods Enzymol., 136: 380-394, incorporated herein in its entirety by reference). Such methods are of use according to the invention. As mentioned below, whole cells are typically cast into agarose for the purpose of delivering intact chromosomal DNA into a matrix suitable for pulsed-field gel electrophoresis or to serve as a “lawn” of host cells that will support bacteriophage growth prior to the lifting of plaques according to the method of Benton and Davis (see Maniatis et al., 1982, Molecular Cloning: A Laboratory Manual, Cold Spring Harbor Laboratory Press, Cold Spring Harbor, N.Y., incorporated herein in its entirety by reference). In short, electrophoresis-grade agarose (e.g., Ultrapure; Life Technologies/Gibco-BRL) is dissolved in a physiological (isotonic) buffer and allowed to equilibrate to a temperature of 50° C. to 52° C. in a tube, bottle or flask. Cells are then added to the agarose and mixed thoroughly, but rapidly (if in a bottle or tube, by capping and inversion, if in a flask, by swirling), before the mixture is decanted or pipetted into a gel tray. If low-melting point agarose is used, it may be brought to a much lower temperature (down to approximately room temperature, depending upon the concentration of the agarose) prior to the addition of cells. This is desirable for some cell types; however, if electrophoresis is to follow cell lysis prior to covalent attachment of the molecules of the resultant nucleic acid pool to the support, it is performed under refrigeration, such as in a 4° C. to 10° C. ‘cold’ room.

The nucleic acid template and/or primer strand may be positioned randomly on the support or at specific ordered locations on the support. In another embodiment, the surface can be separately spotted with templates and/or primers which are randomly deposited on the support. In yet another embodiment, the surface can be coated with a single template and/or primer sequence. The support can also contain ordered sub-features (“islands”) such as a grid of 20×20 micron inkjet deposited 5′-anchored oligos separated by 10 micron borders having no oligos, such that the randomly deposited templates form polonies over the ordered islands. As used herein, the terms “randomly-patterned” or “random” refer to a stochastic, non-ordered, non-Cartesian distribution (in other words, not arranged at pre-determined points along the x- and y axes of a grid or at defined ‘clock positions’, degrees or radii from the center of a radial pattern) of nucleic acid molecules over a support, that is not achieved through an intentional design (or program by which such a design may be achieved) or by placement of individual nucleic acid features. Such a “randomly-patterned” or “random” array of nucleic acids may be achieved by dipping, wicking (capillary wetting), dropping, spraying, plating, flowing, squeegeeing or otherwise spreading a solution, emulsion, aerosol, vapor or dry preparation comprising a pool of nucleic acid molecules onto a support and allowing the nucleic acid molecules to settle onto the support without intervention in any manner to direct them to specific sites thereon.

The nucleic acid template strands may be applied to the substrate by any means known to those skilled in the art. The nucleic acid template strands may also be immobilized to the support by any means known to those skilled in the art including covalent linkage between a nucleic acid molecule and a support matrix or steric hindrance between a nucleic acid molecule and a support matrix.

The nucleic acid template strands may be positioned on a substrate to form an array with the nucleic acid template strands being distanced from one another sufficient to permit the identification of discrete features of the array. As used herein, the term “feature” refers to each nucleic acid sequence occupying a discrete physical location on the array; if a given sequence is represented at more than one such site, each site is classified as a feature. In this context, the term “nucleic acid sequence” may refer either to a single nucleic acid molecule, whether double or single-stranded, to a “clone” of amplified copies of a nucleic acid molecule present at the same physical location on the array.

Pools of nucleic acid molecules are applied directly to the support medium. Alternatively, they are cloned into nucleic acid vectors. For example, pools composed of fragments with inherent polarity, such as cDNA molecules, are directionally cloned into nucleic acid vectors that comprise, at the cloning site, oligonucleotide linkers that provide asymmetric flanking sequences to the fragments. Upon their subsequent removal via restriction digestion with enzymes that cleave the vector outside both the cloned fragment and linker sequences, molecules with defined (and different) sequences at their two ends are generated. By denaturing these molecules and spreading them onto a support to which is covalently bound oligonucleotides that are complementary to one preferred flanking linker, the orientation of each molecule in the array is determined relative to the surface of the support. Such a polar array is of use for in vitro transcription and/or translation of the array or any purpose for which directional uniformity is preferred.

Affixing or immobilizing nucleic acid molecules to the substrate is performed using a covalent linker that is selected from the group that includes oxidized 3-methyl uridine, an acrylyl group and hexaethylene glycol. In addition to the attachment of linker sequences to the molecules of the pool for use in directional attachment to the support, a restriction site or regulatory element (such as a promoter element, cap site or translational termination signal), is, if desired, joined with the members of the pool. Linkers can also be designed with chemically reactive segments which are optionally cleavable with agents such as enzymes, light, heat, pH buffers, and redox reagents. Such linkers can be employed to pre-fabricate an in situ solid-phase inactive reservoir of a different solution-phase primer for each discrete feature. Upon linker cleavage, the primer would be released into solution for PCR, perhaps by using the heat from the thermocycling process as the trigger.

It is also contemplated that affixing of nucleic acid molecules to the support is performed via hybridization of the members of the pool to nucleic acid molecules that are covalently bound to the support.

Immobilization of nucleic acid molecules to the support matrix according to the invention is accomplished by any of several procedures. Direct immobilizing via the use of 3′-terminal tags bearing chemical groups suitable for covalent linkage to the support, hybridization of single-stranded molecules of the pool of nucleic acid molecules to oligonucleotide primers already bound to the support, or the spreading of the nucleic acid molecules on the support accompanied by the introduction of primers, added either before or after plating, that may be covalently linked to the support, may be performed. Where pre-immobilized primers are used, they are designed to capture a broad spectrum of sequence motifs (for example, all possible multimers of a given chain length, e.g., hexamers), nucleic acids with homology to a specific sequence or nucleic acids containing variations on a particular sequence motif. Alternatively, the primers encompass a synthetic molecular feature common to all members of the pool of nucleic acid molecules, such as a linker sequence (see above).

Two means of crosslinking a nucleic acid molecule to a polyacrylamide gel sheet will be discussed in some detail. The first (provided by Khrapko et al., 1996, U.S. Pat. No. 5,552,270) involves the 3′ capping of nucleic acid molecules with 3-methyl uridine. Using this method, the nucleic acid molecules of the libraries of the present invention are prepared so as to include this modified base at their 3′ ends. In the cited protocol, an 8% polyacrylamide gel (30:1, acrylamide: bis-acrylamide) sheet 30 μm in thickness is cast and then exposed to 50% hydrazine at room temperature for 1 hour. Such a gel is also of use according to the present invention. The matrix is then air dried to the extent that it will absorb a solution containing nucleic acid molecules, as described below. Nucleic acid molecules containing 3-methyl uridine at their 3′ ends are oxidized with 1 mM sodium periodate (NaIO₄) for 10 minutes to 1 hour at room temperature, precipitated with 8 to 10 volumes of 2% LiClO₄ in acetone and dissolved in water at a concentration of 10 pmol/μl. This concentration is adjusted so that when the nucleic acid molecules are spread upon the support in a volume that covers its surface evenly and is efficiently (i.e., completely) absorbed by it, the density of nucleic acid molecules of the array falls within the range discussed above. The nucleic acid molecules are spread over the gel surface and the plates are placed in a humidified chamber for 4 hours. They are then dried for 0.5 hour at room temperature and washed in a buffer that is appropriate to their subsequent use. Alternatively, the gels are rinsed in water, re-dried and stored at −20° C. until needed. It is thought that the overall yield of nucleic acid that is bound to the gel is 80% and that of these molecules, 98% are specifically linked through their oxidized 3′ groups.

A second crosslinking moiety that is of use in attaching nucleic acid molecules covalently to a polyacrylamide sheet is a 5′ acrylyl group, which is attached to the primers. Oligonucleotide primers bearing such a modified base at their 5′ ends may be used according to the invention. In particular, such oligonucleotides are cast directly into the gel, such that the acrylyl group becomes an integral, covalently bonded part of the polymerizing matrix. The 3′ end of the primer remains unbound, so that it is free to interact with, and hybridize to, a nucleic acid molecule of the pool and prime its enzymatic second-strand synthesis.

Alternatively, hexaethylene glycol is used to covalently link nucleic acid molecules to nylon or other support matrices (Adams and Kron, 1994, U.S. Pat. No. 5,641,658). In addition, nucleic acid molecules are crosslinked to nylon via irradiation with ultraviolet light. While the length of time for which a support is irradiated as well as the optimal distance from the ultraviolet source is calibrated with each instrument used due to variations in wavelength and transmission strength, at least one irradiation device designed specifically for crosslinking of nucleic acid molecules to hybridization membranes is commercially available (Stratalinker, Stratagene). It should be noted that in the process of crosslinking via irradiation, limited nicking of nucleic acid strands occurs. The amount of nicking is generally negligible, however, under conditions such as those used in hybridization procedures. In some instances, however, the method of ultraviolet crosslinking of nucleic acid molecules will be unsuitable due to nicking Attachment of nucleic acid molecules to the support at positions that are neither 5′-nor 3′-terminal also occurs, but it should be noted that the potential for utility of an array so crosslinked is largely uncompromised, as such crosslinking does not inhibit hybridization of oligonucleotide primers to the immobilized molecule where it is bonded to the support. The production of ‘terminal’ copies of an array of the invention, i.e., those that will not serve as templates for further replication, is not affected by the method of crosslinking. In situations in which sites of covalent linkage are, preferably, at the termini of molecules of the array, crosslinking methods other than ultraviolet irradiation are employed.

According to an alternate embodiment, double-stranded DNAs are deposited by evaporation or other means on a planar surface, trapped in a polymerized gel as a random coil, extended randomly, extended as aligned arrays, or collapsed. A DNA molecule, when it is in solution, behaves as a random globular coil, and its statistical spatial residence volume approximates a sphere with a diameter proportional to the square root of the length in base pairs (bp) with greatest density at the sphere center (Cantor et al., supra). The diameter of this residence sphere can be increased in low salt and/or low divalent cation conditions (e.g., 0.001M Tris-EDTA buffer, pH 8.0) when templates need to be spread out for making marker-to-marker length measurements, or decreased in high salt and/or high divalent cation conditions when template compactness and high-density real estate utilization is desired. The DNA can also be stretched by force created during drying (Aston et al. (1999) Trends Biotech. 17:297-302, incorporated herein in its entirety by reference).

Immobilized nucleic acid molecules may, if desired, be produced using a device (e.g., any commercially-available inkjet printer, which may be used in substantially unmodified form) which sprays a focused burst of reagent-containing solution onto a support (see Castellino (1997) Genome Res. 7:943-976, incorporated herein in its entirety by reference). Such a method is currently in practice at Incyte Pharmaceuticals and Rosetta Biosystems, Inc., the latter of which employs “minimally modified Epson inkjet cartridges” (Epson America, Inc.; Torrance, Calif.). The method of inkjet deposition depends upon the piezoelectric effect, whereby a narrow tube containing a liquid of interest (in this case, oligonucleotide synthesis reagents) is encircled by an adapter. An electric charge sent across the adapter causes the adapter to expand at a different rate than the tube, and forces a small drop of liquid reagents from the tube onto a coated slide or other support.

Reagents can be deposited onto a discrete region of the support, such that each region forms a feature of the array. The desired nucleic acid sequence can be deposited as a whole or synthesized drop-by-drop at each position, as is true for other methods known in the art. If the angle of dispersion of reagents is narrow, it is possible to create an array comprising many features. Alternatively, if the spraying device is more broadly focused, such that it disperses nucleic acid synthesis reagents in a wider angle, as much as an entire support is covered each time, and an array is produced in which each member has the same sequence (i.e., the array has only a single feature).

Arrays of both types are of use in the invention. A multi-feature array produced by the inkjet method is used in array templating, as described above. A random library of nucleic acid molecules are spread upon such an array as a homogeneous solution comprising a mixed pool of nucleic acid molecules by contacting the array with a tissue sample comprising nucleic acid molecules, or by contacting the array with another array, such as a chromosomal array or an RNA localization array.

Alternatively, a single-feature array produced by the inkjet method is used by the same methods to immobilize nucleic acid molecules of a library which comprise a common sequence, whether a naturally-occurring sequence of interest (e.g., a regulatory motif) or an oligonucleotide primer sequence comprised by all or a subset of library members, as described herein above.

Nucleic acid molecules which thereby are immobilized upon an ordered inkjet array (whether such an array comprises one or a plurality of oligonucleotide features) are amplified in situ, transferred to a semi-solid support, and immobilized thereon to form a first randomly-patterned, immobilized nucleic acid array which is subsequently used as a template with which to produce a set of such arrays according to the invention, all as described above.

The following examples are provided for illustrative purposes only and are not intended to limit the scope of the invention which has been described in broad terms above.

### Example I

### The Flip-Recombinase Yielding Two Bits

At least 12 combinations of in vivo memory types can be generated including the following features: 1) continuous or pulsed by 2) reversible or permanent by 3) additive or log-scale or mixed. The first type in each would be the default always-on, reversible, additive (until saturated) flipping system. The term “pulsed” means that the recombinase is under control of an external signal including but not limited to, a signal like estrogen or an internal signal like cell-division. Permanent flips can be achieved by the use of an asymmetric recombinase such as lambda int. A roughly log-scale prototype (with a greater dynamic range) may be constructed by having each bit in a series be an increasingly poor substrate for flipping. The top bits would require large concentrations of recombinase to flip while the bottom bits would require very little activity.

In one embodiment a digital counter would be made using Integrase-1 (I1) which requires pulse condition-1 (C1) and flip promoter/terminator-1 transcribing right and blocking from the left (R1).

I1 activity leads only to the first flip and hence promoter-1 pointing left (L1) and inducing an xis-like activity (I2=reverse of I1).

I1 is specific for R1.

I2 is specific for L1 & R2.

I3 is specific for L2 & R3.

I4 is specific for L3 & R4 . . . .

With a starting state of the first line below:

$$\begin{matrix}R 4 > I 4 R 3 > I 3 R 2 > I 2 R 1 > I 1 & 0000 & I 1 active ( once C 1 begins ) \\ R 4 > I 4 R 3 > I 3 R 2 > I 2 < L 1 I 1 & 0001 & I 2 \\ R 4 > I 4 R 3 > I 3 < L 2 I 2 R 1 > I 1 & 0010 & I 1 \\ R 4 > I 4 R 3 > I 3 < L 2 I 2 < L 1 I 1 & 0011 & I 2 \& I 3 \\ R 4 > I 4 < L 3 I 3 R 2 > I 2 R 1 > I 1 & 0100 & I 1 \\ R 4 > I 4 < L 3 I 3 R 2 > I 2 < L 1 I 1 & 0101 & I 2 \\ R 4 > I 4 < L 3 I 3 R 2 > I 2 R 1 > I 1 & 0110 & I 1 \\ R 4 > I 4 < L 3 I 3 < L 2 I 2 < L 1 I 1 & 0111 & I 2 \& I 3 \& I 4 \\ < L 4 I 4 R 3 > I 3 R 2 > I 2 R 1 > I 1 & 1000 & I 1 \\ < L 4 I 4 R 3 > I 3 R 2 > I 2 < L 1 I 1 & 1001 & I 2 \\ etc . … & & \end{matrix}$$

Other versions, which are faster (e.g. steroid or kinase mechanism) or simpler (fewer new recombinases) can be designed based on the guidelines set forth above.

### Example II

### Minigenome Synthesis

According to one aspect of the present invention, large numbers of arbitrarily long DNA segments, i.e. on the order of millions, can be synthesize with a high degree of accuracy. According to one aspect, mask-less photolithography (Singh-Gasson (1999)) and improved photo-phosphoramidites (Beier (2000) Nucleic Acids Res. 28(4):E11) are used. Alternatively, piezoelectric ink jet deposition of conventional phosphoramidites (Hughes (2001)) is used. In addition to permanent 3′ attachment (for use as immobilized hybridization arrays) both methods are designed for release, either all at once or in phases to act as self-primers for PCR. Protocols similar to those for DNA-shuffling (Crameri (1996)) are used to make up to 20 kilobase-sized constructs. Existing biological systems with two-out-of-three repair mechanisms reduce error rates even further. These are assembled by chewing back the 3′ ends of nucleic acid molecules and extension ligating. Final constructs in the 50 to 200 kbp range are size selected and in some cases further combined using homologous recombination (Link (1997); Swaminathan (2001)). Preferred applications include converting the codon usage of whole proteins or pathways (Andre (1998) J. Virol. 72(2): 1497-1503).

In a preferred embodiment, a minigenome is based on Escherichia coli (and four other species). There are 45 modification enzymes for the 31 modified bases in tRNA and rRNA (Khan (1988)). In a preferred embodiment, the number of tRNAs chosen will range from 20 (the minimum to cover the main 20 amino acids) to 46 (the minimum to cover the main 61 codons) to 85 to cover all tRNAs in E. coli. In another embodiment, nucleic acid amplification, such as PCR or isothermal amplification methods, is used to harvest as many adjacent genes as possible without change in codons initially. Rolling Circle Amplification (RCA) can be used for in vitro DNA replication/amplification. The overall replication of the minigenome is determined by the polymerase extension rate and the number of initiation sites. The latter are defined by the number of either primer (or, in the case of isothermal amplification according to WO 00/41524, supra, helicase/primase) binding sites or by nicking sites.

Screening can be accomplished by microscopic quantitation of arrays of known variants or by isolation of amplification products, such as produced by rolling circle amplification or other suitable amplification methods known in the art, from random arrays. In preferred embodiments, the assay is the level of green fluorescent protein (GFP) fluorescence or luciferase luminescence. Physical selection is accomplished by variations on ribosome display (Mattheakis (1994); Hanes (2000)) and puromycin-mRNA display (Hammond (2001); Kurz (2000)). In one embodiment, affinity selection with Nickel columns or antibodies to the epitope fusion proteins is used. The initial set of screens are for GFP, followed by RCA-dependent GFP, and translational production of the polymerase required for RCA & GFP, and then each of the 140 components of the minigenome (typically in naturally adjacent groups of genes from the E. coli genome) to assess whether they aid or inhibit GFP production. Combinations of regulatory elements and genes will be computationally designed for experimental screening based on the above rounds.

The nicking activity of BstNBI and a subset of the primers can be used in constructing the minigenome. With one origin per genome and the normal rate of Bst-Polymerase of 250 by per second, the doubling time of the population of minigenomes would be 7 minutes. If initiating nicks and/or primers occur every 250 bp, then the doubling rate would be one second. These rates go from about 4-fold faster than the fastest rate in bacteria (E. coli) to 800-fold faster. Note that since replication (and competition) is exponential, even 1.01-fold differences on replication rate turn into 2-fold changes in population size after one hundred generations or so, hence such changes are truly enormous. The relevant rate in a Darwinian competition sense will depend on the rate at which the sibling genome complexes typically segregate and express. This will be limited by the protein synthetic rate. The RNA polymerase chosen (from T7) has a rate similar to the DNA polymerase (i.e. 250 nucleotides per minute). The ribosome advances at about 55 nucleotides per minute and the longest gene is 2853 bp, so the expression time is one minute plus folding/assembly time.

The replicating units form groups of related sibling molecules that are called “polonies” for polymerase-colonies. The boundaries of these need to be rigid enough to keep the polonies separate long enough to have a competitive Darwinian advantage, yet flexible enough to allow rapid growth. They also need to pass food and waste. Preferred methods include (1) isolation by polymer or gels, e.g. acrylamide or agarose (Mitra (1999)) or oil-water emulsion (Tawfik (1998); Ghadessy (2001)). For the porous polymer gels, substrates (amino acids, dNTPs, rNTPs) can be exchanged in and the waste products (dNMPs, rNDPs, PPi, Pi) removed by very rapid dialysis.

### Example III

### Use of Minigenome for 3D Modeling

Mutations can be obtained as part of random drift, subtle adaptations to the in vitro system, and specific adaptations to specific challenges as has been done with tRNA-synthetases (Wang (2001)) and Taq Polymerase (Ghadessy (2001)). The E. coli genome is small enough to allow resequencing. The effects on conserved nucleotides can be computed and the effects on 3D structure can be modeled. One notable advantage of the minigenome of the invention is that in contrast to other genomes where only 10% of the proteins are of known three dimensional structure, the three dimensional structure of 138 of the most essential 140 components of the E. coli replicating system are known through crystallography of closely homologous species.

The structure of the active replication/translation complex may be considerably less compact than a living cell and hence more accessible to microscopic inspection. A 90-kb circle is likely to have a random coil radius of about 1-micron depending on counterions. Immobilizing a flat surface of sequence-specific DNA binding proteins could force the mini genome into a planar or even linear configuration. In the latter case, the maximal distance between two points on an extended circle would be 15 microns easily visible with conventional optics. A variety of sub-50 nm resolution microscopy methods (e.g. atomic force microscopy, near field optics) are applicable and greatly aided computationally by the existence of the component structures.

Stochastic modeling (Gibson 2000, 2000b) and/or related petri nets (Goss (1998); Matsuno (2000)) can be applied to optimize the reproducibility of the complexes as the number of each molecular species approaches a single copy of the expected stoichiometry (typically one to four per complex). In order to obtain the numerous kinetic reaction probabilities and other parameters for these and other systems models to be “over-determined,” the openness of the coupled replication/translation system can be exploited to collect needed data. In one embodiment, several single molecules created by pulsing single eta-DNAPo1 and an RNAPo1-etaDNAPol fusion are sequenced to compare the incorporation variance.

### OTHER EMBODIMENTS

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference in their entirety for all purposes to the same extent as if each individual publication or patent application were specifically and individually indicated to be so incorporated by reference.

What is claimed:

1. A method of using a nucleic acid sequence for encoding information comprising:
a) providing a nucleic acid sequence including a central portion between two binding domains, wherein the central portion is capable of having its position altered on the nucleic acid between two states or eliminated from the nucleic acid when the binding domains are contacted with a site-specific recombinase specific for the binding domains;
b) contacting the two binding domains with the site-specific recombinase; and
c) allowing the site-specific recombinase to alter the position of or eliminate the central portion between the two binding domains.

2. The method of claim 1, wherein the position of the central portion between the two binding domains is irreversibly altered.

3. The method of claim 1, wherein the site-specific recombinase is Flp recombinase.

4. The method of claim 1, wherein the site-specific recombinase is under the control of an inducible promoter.

5. The method of claim 1, wherein the nucleic acid sequence comprises a mini-genome.

6. A method of using an immobilized nucleic acid array for encoding information comprising:
a) attaching a nucleic acid sequence including a central portion between two binding domains, wherein the central portion is capable of having its position altered on the nucleic acid between two states or eliminated from the nucleic acid when the binding domains are contacted with a site-specific recombinase specific for the binding domains on a substrate;
b) contacting the substrate with the site-specific recombinase; and
c) allowing the site-specific recombinase to alter the position of or eliminate the central portion between the two binding domains.

7. A method of analyzing a degree of evolutionary divergence between organisms comprising:
a) providing a first organism expressing a nucleic acid sequence including multiple regions having a central portion between two binding domains, wherein the central portion is capable of having its position altered on the nucleic acid between two states or removed from the nucleic acid when the binding domains are contacted with a site-specific recombinase specific for the binding domains;
b) providing a second organism expressing the nucleic acid sequence in step a);
c) growing the organisms for a specific length of time;
d) analyzing the nucleic acid sequence from the first organism to determine the pattern of central portions states;
e) analyzing the nucleic acid sequence from the second organism to determine the pattern of central portion states; and
f) comparing the pattern of the central portion states to determine the degree of evolutionary divergence between the organisms.

8. The method of claim 7, wherein three or more organisms are compared.

9. The method of claim 7, wherein the position of the central portion between the two binding domains is irreversibly altered.

10. The method of claim 7, wherein the nucleic acid sequence comprises a mini-genome.

11. A method of recording transcriptional activation of a nucleic acid sequence comprising:
a) providing a nucleic acid sequence including one or more regions having a central portion between two binding domains, wherein the central portion is capable of having its position altered on the nucleic acid between two states or removed from the nucleic acid when the binding domains are contacted with a site-specific recombinase specific for the binding domains;
b) providing a nucleic acid encoding the site-specific recombinase under the control of an inducible promoter; and
c) inducing the promoter;
d) wherein promoter induction results in the central portion between the two binding domains having its position altered or removed.

12. A method of synthesizing an array of nucleic acids on a substrate comprising:
a) providing a substrate;
b) placing a plurality of nucleic acid primers on the substrate;
c) contacting the primers with an error-prone polymerase and a reversible chain terminating nucleotide, wherein the polymerase adds the nucleotide to the primer;
d) exposing a selected region to an effector that modifies the nucleotide such that it is not a chain terminating nucleotide;
e) repeating steps c)-d).

13. The method of claim 12, wherein reversible chain terminating nucleotide is photo-reversible.

14. The method of claim 12, wherein the effector is light.

15. The method of claim 12, wherein the array is analyzed by polony fluorescent in situ sequencing.

16. The method of claim 12, wherein the array is analyzed by rolling circle amplification.

17. A method of synthesizing an array of nucleic acids on a substrate comprising:
a) providing a substrate;
b) placing a plurality of nucleic acid primers on selected regions of the substrate;
c) contacting the primers with an error-prone polymerase and a reversible chain terminating nucleotide, wherein the polymerase adds the nucleotide to the primer;
d) exposing the substrate to an effector that modifies the nucleotide such that it is not a chain terminating nucleotide; and
e) repeating steps c)-d).

18. The method of claim 17, wherein reversible chain terminating nucleotide is photo-reversible.

19. The method of claim 17, wherein the effector is light.

20. The method of claim 17, wherein the array is analyzed by polony fluorescent in situ sequencing.

21. The method of claim 17, wherein the array is analyzed by rolling circle amplification.

22. A method of using a nucleic acid sequence for encoding sensor information comprising:
a) providing a sensor, wherein the sensor is altered when contacted by a ligand such that the altered sensor can be incorporated into a nucleotide sequence by an error-prone polymerase;
b) contacting the sensor with a ligand to produce an altered sensor;
c) contacting the altered sensor with an error-prone polymerase, wherein the polymerase adds the sensor to the nucleic acid sequence.

23. The method of claim 22, wherein the sensor is a photo-activated nucleotide.

24. The method of claim 22, wherein the ligand is light.

25. The method of claim 22, wherein the ligand is a metabolic product.

26. The method of claim 22, wherein the ligand is a chemical.

27. The method of claim 22, wherein the nucleic acid sequence encoding sensor information is analyzed by polony fluorescent in situ sequencing.

28. The method of claim 22, wherein the nucleic acid sequence encoding sensor information is analyzed by rolling circle amplification.

29. A method of using a nucleic acid sequence for encoding sensor information comprising:
a) providing a nucleic acid sequence encoding an allosteric ribozyme sensor comprising a downstream gene, wherein the ribozyme is triggered to cleave itself out of the nucleic acid sequence when contacted by a ligand such that the downstream gene is translated at increased levels; and
b) contacting the nucleic acid sequence with the ligand such that the ribozyme is triggered to cleave itself.

30. The method of claim 29, wherein the downstream gene encodes green fluorescent protein.

31. The method of claim 29, wherein the downstream gene is translated at decreased levels after ribozyme cleavage.

32. A method for identifying a modified polymerase that incorporates a chain terminating nucleotide into a nucleic acid polymer at greater than wild-type levels comprising:
a) providing a wild-type polymerase;
b) providing at least one modified polymerase;
c) contacting each polymerase with nucleic acid templates;
d) contacting each polymerase with reversible chain terminating nucleotides and allowing the polymerases to add a chain terminating nucleotide to the templates for a specific amount of time;
e) measuring the rate of chain terminating nucleotide incorporation for each polymerase, wherein a modified polymerase capable of incorporating a chain terminating nucleotide will incorporate the nucleotides at a faster rate than the wild-type polymerase; and
f) identifying the modified polymerase.

33. A method for identifying a modified polymerase that incorporates a labeled nucleotide triphosphate into a nucleic acid polymer at greater than wild-type levels comprising:
a) providing a wild-type polymerase;
b) providing at least one modified polymerase;
c) contacting each polymerase with nucleic acid templates;
d) contacting each polymerase with labeled nucleotide triphosphates and allowing the polymerases to add labeled nucleotide triphosphates to the templates for a specific amount of time; and
e) measuring the amount of labeled nucleotide triphosphate incorporation for each polymerase, and identifying a modified polymerase capable of incorporating more labeled nucleotide triphosphate than the wild-type polymerase.
